(19) United States
(12) Patent Application Publication
(10) Pub. No.: US 2024/0130629 A1
(43) Pub. Date: Apr. 25, 2024

(54) ELECTRODE HARNESS FOR USE IN CARRYING OUT ELECTRICAL IMPEDANCE TOMOGRAPHY, A SYSTEM AND A METHOD

(71) Applicant: CYQIQ LTD, London (GB)

(72) Inventor: David Holder, London (GB)

(21) Appl. No.: 18/005,513

(22) PCT Filed: Jul. 8, 2021
(86) PCT No.: PCT/GB2021/051755

§ 371 Date: Jan. 13, 2023

(30) Foreign Application Priority Data
Jul. 16, 2020 (GB) 2011078.9

(51) Int. Cl. A61B 5/0536; A61B 5/00

(52) U.S. Cl. CPC A61B 5/0536; A61B 5/0002; A61B 5/0024; A61B 5/6805; A61B 5/742; A61B 5/7475; A61B 2503/40; A61B 2562/046

(57) ABSTRACT

An electrode harness for use in carrying out Electrical Impedance Tomography (EIT) on a human or animal subject is disclosed. The electrode harness includes an electrode support a plurality of electrodes on the electrode support for contacting a surface of an external body part of the subject, a plurality of connectors, each connector coupled to a respective electrode of the plurality of electrodes, and a control unit. The control unit includes an interrogator for coupling to the electrodes via the connectors, the interrogator being configured to selectively provide electrical signals to multiple groups of electrodes of the plurality of electrodes and for measuring, in response to the provided electrical signals, electrical response signals at at least some electrodes of the plurality of electrodes, and communication circuitry for transmitting information associated with the electrical response signals to an external computing device. A system and method are also disclosed.

### FIELD OF THE DISCLOSURE

This disclosure relates to an electrode harness for use in carrying out Electrical Impedance Tomography (EIT) on a human or animal subject. A system and a method for determining electrical properties relating to part of a human or animal subject using Electrical Impedance Tomography (EIT) is also disclosed.

### BACKGROUND OF THE DISCLOSURE

Electrical impedance tomography (EIT) is a medical imaging method which enables reconstructed tomographic images of the internal impedance of the subject to be obtained with a ring or array of external electrodes placed around the body part of interest. Typically, a small safe insensible electric current is applied to a pair of external electrodes and voltages are then collected at all other electrodes. An alternating current is applied, usually at about 50 kHz. The impedance is calculated using ohm's law from the applied constant current and recorded voltage and demodulation of the recorded voltage signals. Multiple such transfer impedance measurements are made rapidly using an electronic multiplexer switch to permit the addressing of different electrode pairs. These transfer impedances are then reconstructed into a tomographic image using advanced inverse mathematics. Some known techniques use a computer model with the appropriate geometry for the body parts of interest and electrode placements for the reconstruction. It may be a simple geometric model such as a cylinder for the chest or may be more anatomically realistic using a method such as a finite element mesh (FEM) model derived from the segmenting of an MRI or CT of the body part of interest. Research into the application of EIT is ongoing and includes imaging lung function (e.g. to monitor lung ventilation), cardiac function, brain function, breast cancer, stomach emptying, and bladder filling.

The principal research and commercial interest in the development of biomedical EIT has been in its use in imaging lung function. The impedance of the lungs changes as air is drawn into them during respiration which yields clear images which vary with ventilation and are considered to be accurate in revealing regional ventilation in the lungs down to the level of lobar segments. However, current commercial systems are typically based on a system developed in the 1980s in Sheffield which is termed the “Sheffield Mark 1 applied potential tomography system” and is described in a paper by B H Brown and A D Seagar entitled ‘The Sheffield data collection system’ Clinical Physics and Physiological Measurement, 1987, Vol. 8, Suppl. A, 91-97. The Sheffield Mark one system employs a single ring of 16 electrodes placed around the chest. This introduces errors into the reconstructed images because current in reality travels in three-dimensional throughout the chest but this method assumes that it is a two-dimensional problem. Some reconstruction methods use an internal computer model which is itself only two-dimensional; others may have a three-dimensional model but employ the real electrode geometry of a single ring.

The electronic hardware used for collection of data (e.g. impedance measurements) for EIT has typically been constructed with discrete electronic components and so it is a benchtop system typically about the size of a video recorder. The system contains the essential elements of a constant current or voltage source, electronic multiplexer to select different electrode combinations, recording electronics usually in the form of instrumentation amplifiers and a controlling microcontroller with battery and communication capability. Such electronic hardware is therefore bulky and expensive to produce which makes it less attractive for widespread use outside the research environment. Moreover, the electronic hardware needed for collection of the impedance measurements is usually connected to electrodes by leads approximately 50 cm long. With data collection for currents applied at 50 kHz, these leads may introduce errors into measurement through stray capacitance.

It is desirable to provide an arrangement for collection of data for EIT that is not bulky or expensive to produce and which avoids long leads between the electrodes and data collection circuitry.

### SUMMARY

In accordance with different aspects of the invention, there are provided an electrode harness for use in carrying out Electrical Impedance Tomography (EIT), a system, and a method for determining electrical properties relating to part of a human or animal subject using Electrical Impedance Tomography (EIT) as recited in the accompanying claims.

The configuration of the electrode harness in accordance with the disclosure provides a low cost, easy to use electrode harness for use in performing EIT which can therefore be used by individual consumers.

In an example arrangement, the control unit is integrated with (mounted or disposed on or within) the electrode support. By integrating or disposing the electrodes on the electrode support of the electrode harness and integrating or disposing the control unit with the electrode support, the electrode harness provides a compact arrangement for collecting data (e.g. the measured electrical response signals) for EIT processing without the need for the data collecting electronic circuitry to be separate to the electrode harness and without the need for long wires to connect the electrodes to the data collecting electronic circuitry.

In an example arrangement, the plurality of electrodes may be disposed on the electrode support such that, in use (for example, when the electrode harness is mounted on the subject), the plurality of electrodes are arranged to form a plurality of rings (for example, two, three, four, five or more rings) around the electrode support such that the plurality of electrodes surround the external body part of the subject and with each ring comprising more than one electrode. Arranging the plurality of electrodes on the electrode support such that the plurality of electrodes surround the external body part of the subject can deliver much higher resolution and accuracy for Er processing than a single ring of electrodes.

In an example arrangement, the electrode support may comprise or be formed from elasticated or stretchable material, such as for example, perforated silicone rubber or other synthetic breathable material. With the electrode support made from elasticated or stretchable material, when the electrode harness is mounted on subject, the electrode support extends around the external body part of the subject, and the elasticated/stretchable material of the electrode support helps to provide good mechanical contact of all electrodes to the surface of the subject.

In an example arrangement, the electrodes may be integral with the electrode support. For example, the electrodes may be integrated into the material which forms the electrode support. This helps to provide a low cost electrode harness with integrated electrodes for contacting the surface of the subject.

In an example arrangement in which the electrode support may comprise or be formed from elasticated or stretchable material and the electrodes are self-abrading electrodes, such a configuration for the electrode harness helps for the plurality of electrodes to be rapidly and easily applied to the surface of the subject (e.g. the skin) without preparation of the skin which makes such an electrode harness easy to use.

### BRIEF DESCRIPTION OF THE DRAWINGS

An electrode harness for use in carrying out Electrical Impedance Tomography (EIT), a system and a method for determining electrical properties relating to part of a human or animal subject using Electrical Impedance Tomography (Err), in accordance with the disclosure, will now be described, by way of example only, with reference to the accompanying drawings in which:

FIG. 1 is a schematic diagram of an example system comprising an electrode harness for use in carrying out Electrical Impedance Tomography (EIT) and an external computing device;

FIGS. 2A and 2B are schematic diagrams of front and back views of an example electrode harness for use in carrying out Electrical Impedance Tomography (EIT) for a male subject;

FIGS. 3A and 3B are schematic diagrams of front and back views of an example electrode harness for use in carrying out Electrical Impedance Tomography (EIT) for a female subject;

FIGS. 4A and 4B are schematic diagrams of an example implementation of electrodes for an electrode harness;

FIG. 4C is a photograph of a top perspective view of an example arrangement for one of the electrodes shown in FIG. 4A;

FIG. 5 is a schematic diagram of another example implementation of electrodes for an electrode harness;

FIG. 6 is a block schematic diagram of an example control unit;

FIG. 7 is a schematic diagram showing a user interface of an external computing device and an example of information that may be presented on the user interface;

FIG. 8 is a schematic diagram showing a user interface of an external computing device and an example of information that may be presented on the user interface during an electrode contact quality test operation;

FIG. 9 is a block schematic diagram of an example implementation of an interrogator of the control unit.

### DETAILED DESCRIPTION OF THE DRAWINGS

The disclosure relates to an electrode harness for use in carrying out EIT on a human or animal subject. In use, an electrode support of the electrode harness is mounted on the subject so that a plurality of electrodes disposed on the electrode support contact a surface of an external body part of the subject. The electrode harness further includes a control unit comprising an interrogator which selectively provides (or applies) electrical signals to multiple groups of electrodes of the plurality of electrodes and measures, in response to the provided electrical signals, electrical response signals at at least some electrodes of the plurality of electrodes. The electrical response signals may be measured at at least some other electrodes of the plurality of electrodes (e.g. at different electrodes to those to which the electrical signals are provided or may be some or all of the same electrodes to which the electrical signals are provided). The electrical response signals (in combination with the provided electrical signals) represent impedance responses or impedance measurements due to parts of the body within the external body part and using Electrical Impedance Tomography (EIT) reconstruction, electrical properties of the parts of the body within the external body part may be determined. For example, a tomographic image and/or other data relating to the electrical properties of the parts of the body within the external body part may be determined. From the tomographic image and/or other data relating to the electrical properties, functioning or activity of the parts of the body within the external body part may be measured and monitored.

The external body part may be a chest, head or other external body part of the subject for which EIT is to be carried out. The subject may be a human (or any other mammal) or an animal. If the external body part is a chest and the electrode harness is mounted on the chest of a subject, by carrying out EIT using the electrode harness, electrical properties of the heart and lungs may be determined from the impedance measurements such that functioning of the heart and lungs (such as heart rate, blood pressure, respiratory rate, oxygen saturation or other lung, cardiac or cardiovascular functions) within the chest may be measured and monitored. If the external body part is a head and the electrode harness is mounted on the head of a subject, by carrying out EIT using the electrode harness, electrical properties of the brain may be determined from the impedance measurements and used for different brain applications including: 1) Functional activity measuring pathways in the brain over milliseconds as ion channel open in nervous tissue; 2) Imaging in stroke or head injury over a few minutes; and 3) Imaging changes in blood flow or cell swelling over seconds following activity.

As will be discussed further below, the electrical properties determined by carrying out EIT reconstruction may be provided to an external computing device and information relating to the electrical properties presented to a user on a user interface of the external computing device. The information presented to the user on the user interface may be for health and/or fitness monitoring of the subject by a user (e.g. the subject). Alternatively, the information presented to the user on the user interface may be for remote monitoring of the subject by a user who is a medical care provider (e.g. in telemedicine for diagnosis and monitoring of common health conditions and emergencies). The EIT reconstruction may be performed at the external computing device based on information associated with the electrical response signals and provided electrical signals communicated to the external computing device (over wireless or wired links) from the electrode harness or the EIT reconstruction may be performed at the control unit of the electrode harness and the electrical properties determined from the EIT reconstruction communicated to the external computing device (over wireless or wired links) from the electrode harness.

In the following description, reference will be made to carrying out EIT on the chest or thorax of a human subject (i.e. the external body part is a chest or thorax cavity of a person/subject) by mounting an electrode harness on a person/subject such that the electrodes are in contact with the chest of the person/subject. For simplicity, the terms chest and subject will be used hereinafter. It will however be appreciated that it is not intended that the invention as defined in the appended claims, nor the following disclosure, is limited to carrying out EIT on a chest nor only to human subjects.

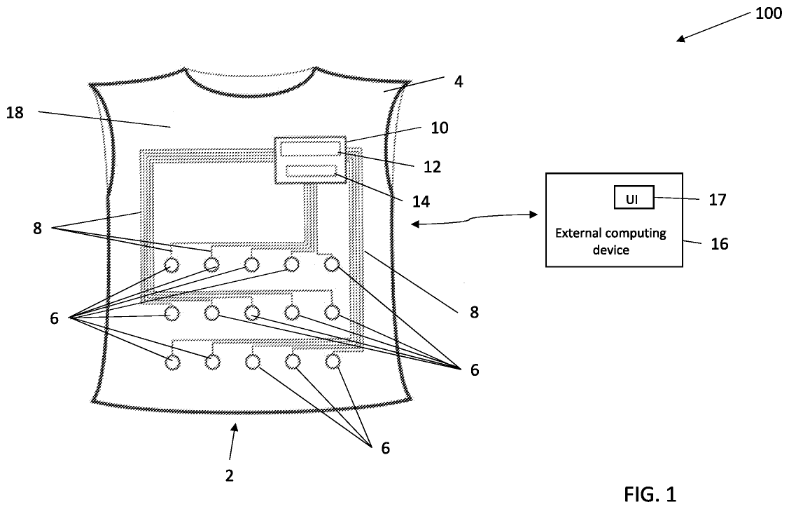

With reference firstly to FIG. 1, an example system 100 for determining electrical properties within a chest of a subject using EIT comprises an electrode harness 2 for use in carrying out EIT, and an external computing device 16 communicably coupled to the electrode harness 2.

The electrode harness 2 comprises an electrode support 4, a plurality of electrodes 6 disposed on the electrode support 4 for contacting a surface of the chest of the subject and a plurality of connectors 8. The connectors 8 may be disposed on the electrode support 4. Each connector 8 of the plurality of connectors is coupled to a respective electrode of the plurality of electrodes 6. The surface of the chest is the skin of the chest. In the case of carrying out EIT on a head, the surface is the scalp of the head and when the electrode harness is mounted on the head, the plurality of electrodes are in contact with the scalp.

The electrode harness 2 further comprises a control unit 10 comprising an interrogator 12 for coupling to the plurality of electrodes 6 via the plurality of connectors 8 (for simplicity FIG. 1 shows the connectors being coupled to the control unit 10) and communication circuitry 14. The interrogator 12 is configured to selectively provide electrical signals to multiple groups of electrodes of the plurality of electrodes 6 and to measure, in response to the provided electrical signals, electrical response signals at at least some electrodes of the plurality of electrodes 6. The operation of the interrogator 12 will be discussed in more detail below. The electrical signals may be current signals (e.g. alternating current) and the electrical response signals may be voltage signals or vice versa. In either case, the resulting electrical response signals represent impedance measurements due to the parts of the body within the chest (such as lungs, heart etc). The communication circuitry 14 transmits information associated with the electrical response signals to the external computing device 16.

In an example arrangement in accordance with an embodiment, the control unit 10 is integrated with (mounted or disposed on or within) the electrode support 4. For example, all the components of the control unit 10 may be mounted on a circuit board and integrated with (mounted or disposed on or within) the electrode support 4. By integrating or disposing the electrodes on the electrode support 4 of the electrode harness 2 and integrating or disposing the control unit 10 with the electrode support 4, the electrode harness 2 provides a compact arrangement for collecting data (e.g. the measured electrical response signals) for ETT processing without the need for the data collecting electronic circuitry to be separate to the electrode harness and without the need for long wires to connect the electrodes to the data collecting electronic circuitry.

In an alternative arrangement, the control unit 10 may be separated into a low cost standalone box with a rechargeable battery and mains power. A connector would lead to a lead from the harness electrode 2.

The external computing device 16 may be a smart phone, a mobile phone, portable telephone, tablet device, wireless video or multimedia device, a personal digital assistant (PDA), a laptop computer, a personal computer, a wired device or any other similar computing device. Communications between the communication circuitry 14 of the control unit 10 and the external computing device 16 may be over wireless communication links and/or a wired connection. The wireless communication links may include one or more wireless links implemented using any suitable communication protocol or standard, such as WiFi, Bluetooth, Zigbee, GSM, UMTS, 5G, Ultra-wideband (UWB). The communication circuitry may therefore comprise a wireless communication interface for supporting wireless communication over wireless links according to the relevant communication protocol or standard (e.g. the wireless communication interface includes a wireless transmitter, wireless receiver, antenna, etc. for WiFi, or Bluetooth or Zigbee etc.). The communication circuitry may additional or alternatively include an interface for a wired connection (e.g. for a USB connector or Ethernet connector). The external computing device may comprise a user interface 17, such as a display. The electrical properties determined by carrying out EIT using the electrode harness 2 may be provided to the external computing device 16 and presented to a user on the user interface 17. The user interface will typically be a display. The information presented to the user on the user interface may be used by the user for health and/or fitness monitoring. Alternatively, the information presented to the user on the user interface may used by a user who is a medical care provider for remote monitoring in telemedicine for diagnosis and monitoring of common health conditions and emergencies.

The plurality of electrodes 6 may be disposed on the electrode support such that, in use (for example, when the electrode harness is mounted on the subject), the plurality of electrodes are arranged to form a plurality of rings (for example, two, three, four, five or more rings) around the electrode support such that the plurality of electrodes 6 surround the external body part of the subject and with each ring comprising more than one electrode. FIG. 1 shows three rings of electrodes disposed on a front portion 18 of the electrode support 4 and with each ring comprising four electrodes but does not show the electrodes on a back portion of the electrode support 4. The number of rings and number of electrodes shown in FIG. 1 is for illustrative purposes only. The number of rings and the number of electrodes in each ring may vary and will be determined in part by the external body part that is to be imaged using ETT (e.g. due to the shape and/or size and/or anatomy of the external body part). In an example arrangement for imaging a chest of a human subject, the plurality of electrodes is arranged in three or four rings around the electrode support 4 and each ring comprises sixteen electrodes (i.e. the plurality of electrodes include thirty-two electrodes E0-E31). Alternatively, three or four rings with each ring comprising eight electrodes may be used. Arranging the plurality of electrodes 6 in a plurality of rings around the electrode support 4 such that the plurality of electrodes 6 surround the external body part of the subject can deliver much higher resolution and accuracy than a single ring of electrodes.

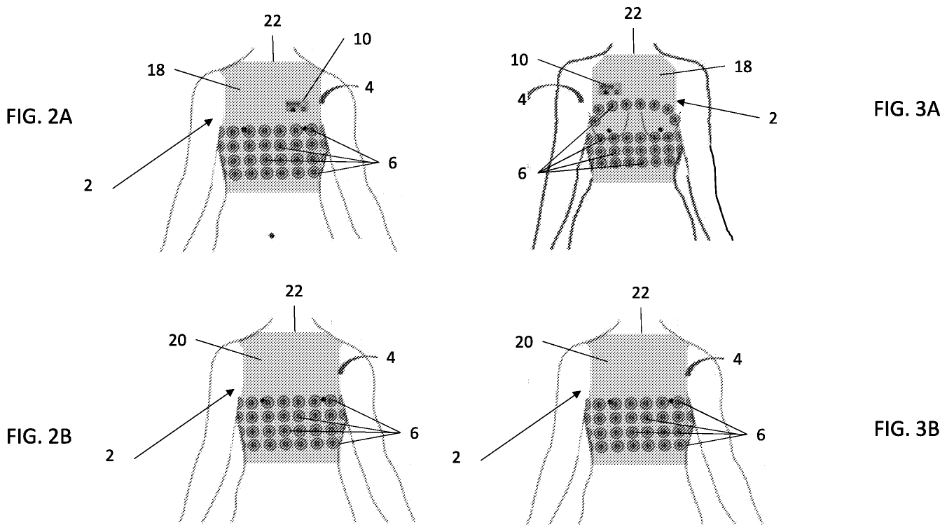

FIGS. 2A and 2B are schematic diagrams showing an example arrangement of the plurality of electrodes 6 of the electrode support 4 for carrying out EIT on a chest of a male human subject when the electrode harness 2 is mounted on the male human subject. FIG. 2A shows the front portion 18 of the electrode support 4 and FIG. 2B shows a back portion of the electrode support. Like components to those of FIG. 1 are referenced by the same reference numeral. In the example arrangement shown in FIGS. 2A and 2B, the electrode support 4 comprises the front portion 18 and back portion 20 with the front portion being connected to the back portion with a head opening 22 therebetween and openings for the subject's arms. The plurality of electrodes 6 are disposed on the front portion 18 and the back portion 20 of the electrode support 4. The plurality of electrodes 6 are arranged in a plurality of rings (four in the example shown in FIGS. 2A and 2B) extending around the front 18 and back 20 portions in a direction substantially perpendicular to a line extending through a centre of the head opening 22. In FIG. 2A, the control unit 10 is shown integrated with (or mounted on) the front portion 18 of the electrode support 4 and although not shown in FIGS. 2A and 2B each connector of the plurality of connectors extend from a respective electrode to the interrogator of the control unit 10 over the front portion 18 and/or back portion 20. Similarly, FIGS. 3A and 3B are schematic diagrams showing an example arrangement of the plurality of electrodes 6 of the electrode support 4 for carrying out EIT on a chest of a female human subject when the electrode harness 2 is mounted on the female human subject. FIG. 3A shows the front portion 18 of the electrode support 4 and FIG. 3B shows a back portion 20 of the electrode support. Like components to those of FIG. 1 are referenced by the same reference numeral. In the example arrangement shown in FIGS. 3A and 3B, the electrode support 4 comprises the front portion 18 and back portion 20 with the front portion being connected to the back portion with a head opening 22 therebetween and openings for the subject's arms. The plurality of electrodes 6 are disposed on the front portion 18 and the back portion 20 of the electrode support 4. The plurality of electrodes 6 are arranged in a plurality of rings (four in the example shown in FIGS. 3A and 3B) extending around the front 18 and back 20 portions in a direction substantially perpendicular to a line extending through a centre of the head opening 22. In FIG. 3A, the control unit 10 is shown integrated with (or mounted on) the front portion 18 of the electrode support 4 and although not shown in FIGS. 3A and 3B each connector of the plurality of connectors extend from a respective electrode to the interrogator of the control unit 10 over the front portion 18 and/or back portion 20. In alternative arrangements, the control unit 10 may instead be integrated with or mounted on another part of the electrode support, such as the back portion of the electrode support 4 or a side portion or a shoulder portion.

For carrying out BIT on the head, the electrode support 4 could be in the shape of a bathing cap, with 32 similar electrodes placed widely over the scalp in a modified 10-20 standard EEG format.

Each electrode of the plurality of electrodes 6 is configured to be a self-abrading electrode.

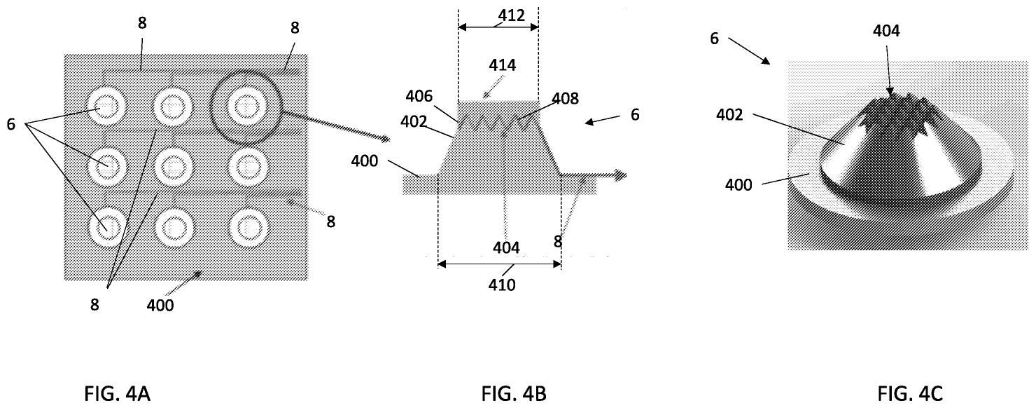

Referring now also to FIGS. 4A, 4B and 4C which show an example construction for the electrodes. Like components to those of the previous figures are referenced by the same reference numeral. Only a couple of electrodes 6 are shown in FIG. 4A. FIG. 4B shows a cross-section of one electrode 6 of the couple of electrodes shown in FIG. 4A. FIG. 4C shows a photograph of a top perspective view of an example arrangement for one electrode 6 of the couple of electrodes shown in FIG. 4A. As shown in FIG. 4A, the electrodes 6 are disposed on a surface 400 of the electrode support 4 and each one of the electrodes 6 is coupled to a respective connector 8 of the plurality of connectors. The plurality of connectors 8 are also disposed on the surface 400. As shown in FIG. 4B, each electrode 6 comprises an electrode mount 402 extending from the surface 400 of the electrode support 4 to provide an electrode mounting surface 404 at an end 406 furthest from the surface 400. A conductive pad 408 may be formed on at least a portion of the electrode mounting surface 404. The conductive pad 408 is electrically coupled to the respective connector 8. When the electrode harness 2 is mounted on the subject, the conductive pads 408 of the electrodes contact the surface (e.g. skin) of the subject. In the example construction as shown in FIG. 4B, at least a portion of the electrode mounting surface 404 is serrated and the conductive pad 408 is formed on at least a portion of the serrated electrode mounting surface 404. The electrode mount 402 may have a flat conical shaped, and the electrode mounting surface 404 (whether serrated or not) may be at least part of a flat surface of the flat conical shaped electrode mount 402. In an example arrangement as shown in FIG. 4C, the electrode mounting surface 404 may be serrated and may comprise a plurality of parallel rows of serrated edges across the electrode mounting surface 404. In an example implementation, the electrode mount 402 may be 10 mm in diameter (see the reference numeral 410) at a base disposed on the surface 400 of the electrode support 4, 8 mm high (between the base and the electrode mounting surface 404) and the electrode mounting surface 404 may have a diameter of 5 mm (see the reference numeral 412). In the example construction as shown in FIG. 4B, the conductive pad 408 is covered with a conductive adhesive material 414, such as hydrogel. The adhesive material 414 and/or the serrated electrode mounting surface 404 configures the electrodes 6 to be self-abrading and thus, facilitates the application of the electrodes 6 to contact the surface (e.g. skin) of the subject when the electrode harness 2 is mounted on the subject.

To reduce cost, the conductive pads 408 of the electrodes 6 and the connectors 8 (e.g. tracks conducting to the electronics) may be formed at the same time from the same material. For example, the conductive pads 408 of the electrodes 6 and the connectors 8 may be formed by vapour deposition, sputtering or similar thin film methods with a conductive material such as metal. In an example implementation, a low cost conductive material such as aluminium, or tin, or carbon particles or silver is used. Other conductive material deposition methods may be used such as thick film or silk screen printing of conductive dye. Although not shown in the Figures, the connectors 8 may be covered by an insulating layer (not shown), leaving the conductive pads 408 exposed at the ends 406 of the electrodes 6. The insulating layer may also extend over the control unit 10. The connectors 8 (or tracks) may be made of material such as aluminium that oxidises spontaneously to provide an insulting layer while conductive pads 408 of the electrodes 6 are covered with material, such as hydrogel, to prevent this and retain electrical conductivity.

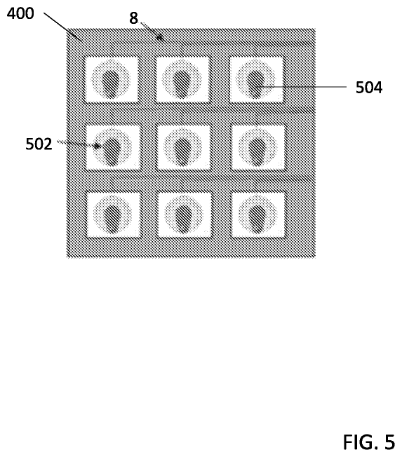

FIG. 5. shows another example construction for the electrodes 6. Like components to those of the previous figures are referenced by the same reference numeral. In FIG. 5, the electrodes are ECG type electrodes 502 disposed on the surface 400 of the electrode support 4 along with the connectors 8. Although not shown in FIG. 5, conductive pads 504 of the ECG type electrodes for contacting the surface of the subject may be mounted on an electrode mount extending from the surface 400 of the electrode support 4 (e.g. similar to the electrode mount 402 of FIG. 4B or 4C). The connectors 8 may be fainted by vapour deposition, sputtering or similar thin film methods with a conductive material such as metal. In an example implementation, a low cost conductive material such as aluminium, or tin, or carbon particles or silver is used. Other conductive material deposition methods may be used such as thick film or silk screen printing of conductive dye. Although not shown in the Figures, the connectors 8 may be covered by an insulating layer (not shown), leaving the ECG type electrodes 502 exposed for contacting the surface (e.g. skin) of the subject. The insulating layer may also extend over the control unit 10. The connectors 8 (or tracks) may be made of material such as aluminium that oxidises spontaneously to provide an insulting layer while conductive pads 408 of the electrodes 6 are covered with material, such as hydrogel, to prevent this and retain electrical conductivity.

The electrode support 4 may comprise or be formed from flexible, elasticated or stretchable material, such as for example, perforated silicone rubber or other synthetic breathable material. The plurality of electrodes 6 (whatever the construction e.g. according to FIGS. 4A and 4B or FIG. 4C or FIG. 5 or some other construction) are disposed on the electrode support 4 such that when the electrode support 4 is mounted on the subject the plurality of electrodes 6 are in contact with the surface of the subject (e.g. in contact with the skin of the chest). The electrode support 4 may be padded in some regions, such as over the sternum, in order to ensure good mechanical contact of all electrodes 6 to the surface of the subject.

To reduce cost, in an example implementation the electrodes 6 are integral with the electrode support 4. For example, the electrodes 6 may be integrated into the material which forms the electrode support 4. If possible, for example, the electrode mounts 402 of FIGS. 4A-4C (and similar electrode mounts for the ECG type electrodes 502 of FIG. 5) may comprise or be formed of the same material which forms the surface of the electrode support 4. For example, when the electrode support 4 is made from silicone rubber which is soft, the electrode mounts 402 are formed from the silicone rubber but since the electrode mounts 402 project from the electrode support 4, they are made from harder material than the electrode support 4. This might be achieved by a suitable process such as heating, laser treatment or chemical process of the silicone rubber.

Instead of forming the connectors 8 by vapour deposition, sputtering or similar thin film methods, other designs may also be used such as carbon loaded rubber or flexible wires embedded in or attached to the flexible electrode support 4. Other conductive material deposition methods may be used such as thick film or silk screen printing of conductive dye.

In order to show the arrangement of the electrodes 6, connectors 8 (in FIG. 1) and control unit 10 integrated with (disposed/mounted on or within) the electrode support 4 in relation to the chest of a human subject when the electrode harness is mounted on the subject, FIG. 1 and FIGS. 2A, 2B, 3A and 3B show an outline of the electrode support 4. It will however be appreciated that as the plurality of electrodes are disposed on the electrode support 4 in order to contact the surface of the chest (e.g. skin) of the human subject, unless the electrode support 4 is made from transparent material, it will not be possible to see the electrodes when looking at the electrode harness 2 mounted on the subject.

With the electrode support 4 made from flexible, elasticated or stretchable material, when the electrode harness 2 is mounted on subject, the electrode support 4 extends around the chest of the subject, and the flexible/elasticated/stretchable material of the electrode support 4 and configuration of the electrodes 6 can provide good mechanical contact of all electrodes 6 to the surface of the subject. Moreover, the flexible electrode support 4 and self-abrading configuration of the electrodes 6 enables the plurality of electrodes 6 to be rapidly and easily applied to the surface of the subject (e.g. the skin) without preparation of the skin. The electrode harness 2 is therefore easy to use.

As discussed above, one or more of several features enable the cost of the electrode harness to be low. The electrode harness could therefore be disposable and single use only or reusable but at low cost. For example, in the case when the electrode harness is reusable, in order to ensure appropriately good electrode performance, additional steps, such as re-gelling of the electrode conductive pads, may be required.

The electrode support 4 may be formed such that front portion 18 connects to the back portion 20 with a head opening 22 and openings for the subject's arms and the subject slips the electrode support 4 over their head to mounting the electrode harness 2 on their chest (due to the flexible/elasticated/stretchable material of the electrode support 4). In other arrangements, a zip or other coupling device at the front or back or side or shoulder of the electrode support 4 may connect securely different portions of the electrode support 4 once the electrode support 4 has been mounted on the subject, with the flexible/elasticated/stretchable material of the electrode support 4 and configuration of the electrodes 6 providing good mechanical contact of the electrodes 6 to the surface of the subject around the external part of the subject.

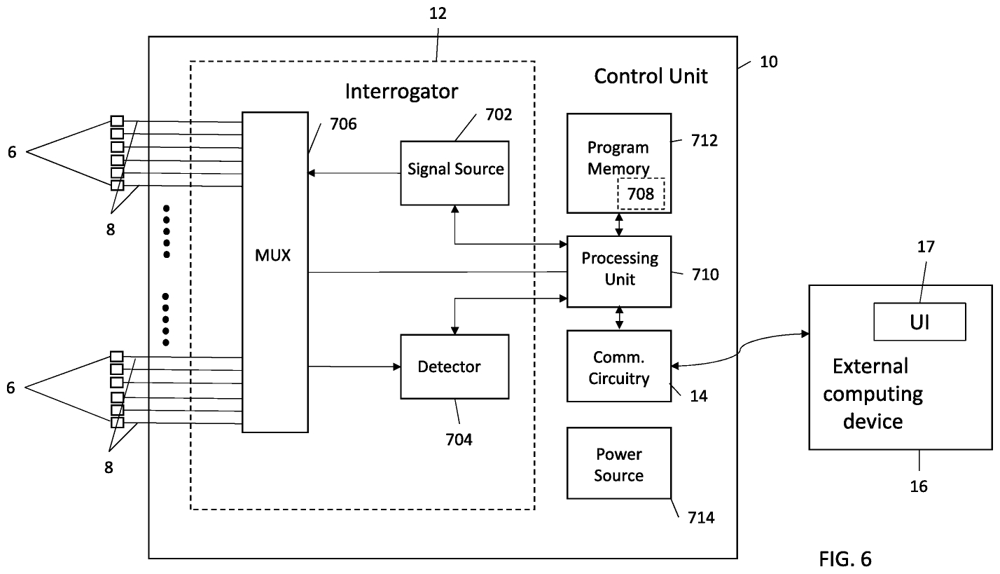

Referring now to FIG. 6 which is a block diagram of an example implementation of a control unit, such as the control unit 10 shown in FIGS. 1, 2A, 2B, 3A and 3B. As will be apparent to a skilled person, FIG. 6 shows only the functional components of an exemplary control unit that are necessary for an understanding of the disclosure. Like components to those of the previous figures are referenced by the same reference numeral.

The control unit 10 comprises the interrogator 12 for coupling to the plurality of electrodes 6 via the plurality of connectors 8 and the communication circuitry 14 for wireless or wired communication with the external computing device 16 as discussed above with reference to FIG. 1.

The interrogator 12 is configured to selectively provide electrical signals to multiple groups of electrodes of the plurality of electrodes 6 and for measuring, in response to the provided electrical signals, electrical response signals at at least some electrodes of the plurality of electrodes 6. The interrogator 12 may comprise a signal source 702 for selectively providing electrical signals to the multiple groups of electrodes and a detector 704 for measuring, in response to the provided electrical signals, and for each group of electrodes of the multiple groups of electrodes, electrical response signals at at least some electrodes of the plurality of electrodes 6. The electrical response signals may be measured at at least some electrodes of the plurality of electrodes 6 (e.g. at different electrodes to those to which the electrical signals are provided and/or may be some or all of the same electrodes to which the electrical signals are provided). The group of electrodes may be a pair of electrodes or four electrodes or other combination of electrodes (e.g. in a desired spatial pattern). Although an electrical signal may be applied to each group of electrodes in turn, with electrical response signals then being recorded for all relevant electrodes (e.g. some or all of the same electrodes to which the electrical signal has been applied and/or some or all of different/other electrodes) before an electrical signal is applied to the next group of electrodes, electrical signals may be provided or applied to multiple groups of electrodes in parallel using multiplexing techniques. Such techniques are well known in the art and are discussed, for example, in WO2009/068961 (the contents of which are incorporated herein in their entirety for all purposes) and other documents, including techniques using frequency division (multiple frequencies at the same time) and code division multiplexing between multiple different groups of electrodes. Phase division multiplexing may also be used. Use of such multiplexing allows a complete set of resulting electrical responses to be collected very rapidly. To implement multiplexing, the interrogator may also comprise a multiplexer or switch 706 for connecting the signal source 702 across multiple groups of electrodes 6 (e.g. for example, the group of electrodes may be a pair of electrodes or four electrodes or other combination of electrodes) in order to apply an electrical signal to or between the electrodes of the multiple groups. The detector 704 is then used to measure resulting electrical response signals at one or more of the plurality of electrodes 6 as connected to the detector 704 by the multiplexer or switch 706.

In an example implementation where the electrode harness 2 comprises a plurality of electrodes 6 disposed on the electrode support 4 such that, in use when the electrode support 4 is mounted on the subject, the plurality of electrodes are arranged in a plurality of rings around the electrode support 4 so as to surround the external body part, with each ring comprising more than one electrode, the interrogator 12 may be configured to measure, in response to electrical signals being provided to electrodes of a particular ring, electrical response signals at electrodes of the particular ring. In other words, each ring of the plurality of rings are treated individually with electrical signals being applied and electrical response signals being measured within each ring. Alternatively, electrical signals may be applied and electrical response signals measured using electrodes across different rings.

Techniques of providing electrical signals and collecting and carrying out EIT reconstruction of the resulting electrical response signals to derive electrical properties (typically corresponding to impedance) within the space surrounded by the electrodes (e.g. to generate a tomographic image or other data relating to the electrical properties) are well known in the art. For example, example techniques are described in WO2009/068961. The EIT reconstruction may be arranged to provide a map or image of the electrical properties across the cross section of the external body part (e.g. within the chest) of the subject or may be arranged to provide the electrical properties at one or more selected points or in one or more selected regions of the external body part (e.g. within the chest). The resulting data may represent the functioning or activity within the external body part at various levels of resolution.

The electrical signals provided by the signal source 702 may be current signals (e.g. alternating current) and the electrical response signals detected by the detector 704 may be corresponding alternating voltage signals. Alternatively, the electrical signals provided by the signal source 702 may be voltage signals and the electrical response signals detected by the detector 704 may be corresponding current signals. The applied electrical signals may be AC or DC but typically, in practice, AC electrical signals are used. In either case, the resulting electrical response signals measured at the electrodes 6, in combination with the electrical signals applied to the electrodes 6, represent impedance responses due to the parts of the body within the chest (such as lungs, heart etc). The impedance responses allow an impedance or conductance map within the plurality of electrodes 6 to be deduced. For example, an EIT reconstruction of the impedance responses can be carried out or performed to derive a tomographic image and/or other data relating to electrical properties within the space surrounded by the plurality of electrodes 6. Typically, the EIT reconstruction uses advanced inverse mathematics and may use a computer model with the appropriate geometry for the body parts of interest and electrode placements. It may be a simple geometric model such as a cylinder for the chest or may be more anatomically realistic using a method such as a finite element mesh (FEM) model derived from the segmenting an MRI or CT of the body part of interest.

The impedance measurements comprise two components from resistance and capacitance; the equivalent resistance due to current flow through capacitance is termed “reactance”. The resistance mainly corresponds to current passing through tissue by ionic conduction in salt solutions, and therefore the extracellular space. The reactance is due to current flow through tissue capacitances, and so mainly is due to current passage through interfaces which store charge, such as cell membranes. Although the reactance can in principle provide useful information, it is usually discarded, as it cannot be measured accurately because of instrumentation errors due to stray capacitance in the electronics and leads.

Typical currents applied to the skin of a human chest are about 1-5 mA at 50 kHz and in typical systems with 16 electrodes placed around the chest may produce up to 50 images per second. Although high quality images are usually collected with current at about 50 kHz, it is also possible to collect reliable images for time difference imaging in the chest at other frequencies generally in the range between 5 kHz and 100 kHz. For frequency difference imaging in the head, frequencies generally in the range between 10 Hz and 10 kHz can be used. For other applications, such as imaging neural circuit activity in the brain frequencies generally in the range between 100 Hz and 10 kHz may be used.

The communication circuitry 14 transmits information associated with the electrical response signals to the external computing device 16 over wireless or wired links. More details of the communication circuitry 14 are discussed above with reference to FIG. 1.

The control unit 10 may also comprise a processing unit 710. The processing unit 710 may be a single processor or may comprise two or more processors carrying out the processing required for the operation of the control unit 10. The control unit 10 also has a program memory 712 in which is stored data and programs containing processor instructions for the operation of the control unit 710. The programs may contain a number of different program elements or sub-routines containing processor instructions for a variety of different tasks for the operation of the control unit, such as for providing control signals to control the generation of the electrical signals by the signal source 702, for providing control signals to control the switching of the multiplexer or switch 706, for controlling communication with the external computing device 16 via the communication circuitry 14. The number of processors and the allocation of processing functions to the processing unit 710 is a matter of design choice for a skilled person. In an implementation which uses a microcontroller, the processing unit 710 and program memory 712 may be integrated into one chip.

The control unit 10 may also comprise a power source 714, such as battery, for providing power to the different components of the control unit 10.

The EIT reconstruction of the impedance responses to derive electrical properties relating to part of the subject within the external body part surrounded by the plurality of electrodes 6 (e.g a tomographic image and/or other data relating to electrical properties) may be performed by the control unit 10 or may be performed by the external computing device 16. When the EIT reconstruction is performed by the control unit 10, the control unit also includes an EIT processing module (e.g. a program stored in the program memory 712 represented by the dotted box 708 in FIG. 6) comprising instructions for carrying out an EIT reconstruction of impedance responses based on the provided electrical signals and the measured electrical response signals to derive electrical properties relating to part of the subject within the external body part. In this case, the information provided to the external computing device 16 by the control unit 10 via the communication circuitry 14 includes the electrical properties derived from the EIT reconstruction. When the EIT processing is performed by the external computing device 16, the external computing device 16 includes an EIT processing module (e.g. a program stored in a program memory (not shown) of the external computing device 16) comprising instructions for carrying out an EIT reconstruction of impedance responses based on the provided electrical signals and the measured electrical response signals to derive electrical properties relating to part of the subject within the external body part. In this case, the information provided by the control unit 10 to the external computing device 16 via the communication circuitry 14 includes information associated with the electrical response signals (e.g. the electrical response signals and the electrical signals provided by the signal source 702 and/or the impedance responses determined based on the provided electrical signals and the measured electrical response signals). In an example arrangement where the external computing device 16 comprises a mobile phone or smart phone or tablet or similar such device, the EIT processing module may be a downloadable application (APP) that can be downloaded (e.g. by the user of the mobile phone or smart phone or tablet or similar such device) from an application server.

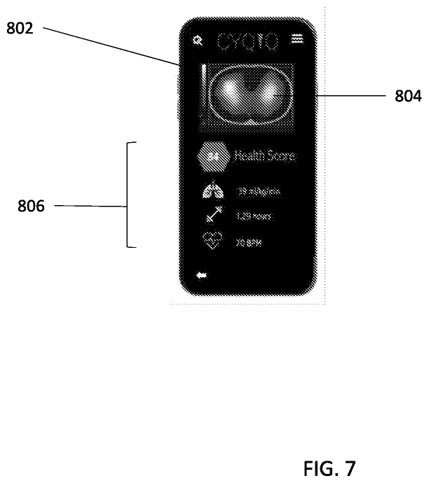

The external computing device 16 comprises a user interface 17. The electrical properties determined by carrying out EIT using the electrode harness 2 may be provided to the external computing device 16 and information relating to the electrical properties presented to a user on the user interface 17. The user interface 17 may include one or more elements such as a key pad, microphone, speaker, display. Typically, the user interface 17 on which information relating to the electrical properties is presented is a display (such as the display 802 of FIG. 7 which shows the external computing device 16 as a smart phone or mobile phone). The information presented may include a tomographic image 804 reconstructed from the impedance responses and/or other data 806 relating to the electrical properties. The other data 806 may include one or more parameters relating to the functioning of part of the subject within the external body part determined by the external computing device 16 based on the derived electrical properties and by extracting additional information from the observed tomographic images and determined electrical properties. For example where the body part is the chest, it is also possible to determine changes in impedance related to blood flowing through the heart and lungs. These changes may be observed with a cycle length of the ECG and provide an indirect index to cardiac output and pulmonary blood flow. Thus, where the external body part is the chest, the one or more parameters may include at least one of the following: lung ventilation, cardiac output, aortic pulse transit time, respiratory and pulse rate. Techniques for determining the other data (such as the one or more parameters) based on the derived electrical properties and by extracting additional information from the observed tomographic images are well known in the field of EIT (see, for example, the book entitled ‘Electrical Impedance Tomography: Methods, History and Applications’ (Series in Medical Physics and Biomedical Engineering) by David. Holder (the contents of which are incorporated herein in their entirety for all purposes). The information presented to the user on the user interface may be for health and/or fitness monitoring of the subject by a user (e.g. the subject). Alternatively, the information presented to the user on the user interface may be for remote monitoring of the subject by a user who is a medical care provider (e.g. in telemedicine for diagnosis and monitoring of common health conditions and emergencies).

It is also noted that the electrical properties derived from the EIT reconstruction may also be relayed to remote stations for expert interpretation and storage.

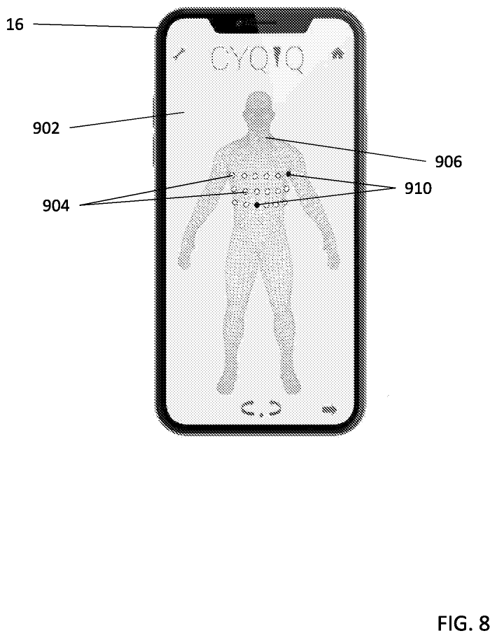

The control unit 10 may be further configured to carry out an electrode contact quality test for each electrode of the plurality of electrodes 6 when the electrode support 4 is mounted on the subject to determine, for each tested electrode, whether a contact impedance between the tested electrode and the surface (e.g. skin) of the external body part of the subject meets a predetermined criteria. and to provide contact quality test result information for transmission by the communication circuitry 14 to the external computing device 6. For example, the control unit 10 may further comprise a contact quality test module (not shown) implemented by a program stored in the program memory 712) to carry out an electrode contact quality test for each electrode of the plurality of electrodes 6 when the electrode support 4 is mounted on the subject. In an implementation where the user interface 17 of the external computing device 6 is a display, the external computing device may display a representation of contact quality of each of the plurality of electrodes based on the contact quality test result information provided by the control unit 10. For example, as shown in FIG. 8, the display 902 of an external computing device 16 (shown as a smart phone or mobile phone) may display representations of each of the plurality of electrodes 6 in the form of circles 904 on the display 902. Like components to those of the previous figures are referenced by the same reference numeral. The image 906 represent the electrodes 6 on the front portion of the electrode support 4. It will be appreciated that a similar image may be displayed on the display 902 for the back portion of the electrode support 4 showing the electrodes 6 on the back portion. Images for the electrodes on the front and back portion may be displayed on the display 902 at the same time. The displayed representations of the electrodes may identify each tested electrode for which it has been determined by the electrode contact quality test performed by the control unit 10 that the contact impedance between the tested electrode and the surface (e.g. skin) of the external body part of the subject does not meet a predetermined criteria. In the example shown in FIG. 8, the tested electrodes that do not meet the predetermined criteria are shown as black circles and designated by reference numerals 910. In the example, shown in FIG. 8, the tested electrodes that do meet the predetermined criteria are shown as white circles. The circles are shown in FIG. 8 as black and white circles for illustrative purposes. In another example, the tested electrodes that do not meet the predetermined criteria may be represented by red circles on the display 902 and the tested electrodes that do meet the predetermined criteria may be represented by green circles on the display 902. Other means for identifying electrodes that do not have sufficiently good contact (e.g. only identifying the tested electrodes that do not meet the predetermined criteria) could be used. Identifying tested electrodes that do not meet the predetermined criteria on a display of the external computing device 16 enables an user to see easily see at a glance if any electrodes had insufficiently good contact. Once a user is alerted to the fact that one or more electrodes do not have sufficiently good contact (e.g. by means of the display on the external computing device 16), the user (e.g. subject) can place their hand over the identified one or more electrodes and rub them in a circular or back-and-forth manner to abrade the skin and decrease the contact impedance to an acceptable level. The mechanical design of the electrode support 4 combined with self-abrading electrodes such as the example electrode constructions described with reference to FIGS. 4A-4C described above or FIG. 5 described above, facilitates this process and ensures that good contact can be obtained between the plurality of electrodes 6 and the skin of the external body part. For example, in the example electrode construction as shown in FIGS. 4A-4C, when the user rubs over the poor contact electrodes in a circular or back-and-forth motion, this would allow the serrated inner surface (electrode mounting surface 404) of the electrodes to abrade the skin and so reduce impedance until at an acceptable level for the contact impedance is achieved as may then be indicated on the display 902 of the external computing device 16 following a subsequent electrode contact quality test performed for each electrode of (e.g. by a white circle representation of the relevant electrodes). For those tested electrodes which are determined to not meet the predetermined criteria (even after rubbing by the user/subject as discussed above), these can be identified and any data from these tested electrodes can be excluded from the ETT reconstruction.

In an example arrangement, the predetermined criteria is met when the measured contact impedance is equal or less than a threshold impedance and the predetermined criteria is not met when the measured contact impedance is greater than the threshold impedance. The threshold impedance may be 5 kohm at 10 Hz. In another example arrangement, the impedance threshold may be 1 kohm at 10 Hz or less than 1 kohm at 10 Hz. In an example arrangement, when the electrode support 4 is mounted on the subject, the electrode contact quality test (performed by the control unit 10, for example, under control of the contact quality test module (not shown) implemented by a program stored in the program memory 712) comprises for each tested electrode: measuring the contact impedance of the tested electrode; comparing the measured contact impedance with a threshold impedance; when the measured contact impedance is greater than the threshold impedance, identifying the tested electrode as having a poor contact quality. When at least one tested electrode of the plurality of electrodes is identified as having a poor contact quality, the control unit 10 is configured to provide contact quality test result information identifying the at least one tested electrode having poor contact quality for transmission by the communication circuitry 14 to the external computer device 16. The measuring the contact impedance of the tested electrode may comprise, for example, providing electrical signals from the current source 702 to all the plurality of electrodes and measuring the electrical response signal on each one (i.e. each one is then a tested electrode) of the plurality of electrodes to determine the effective contact impedance of the tested electrode. The threshold impedance may be 5 kohm with the applied electrical signals at 10 Hz. In another example arrangement, the impedance threshold may be 1 kohm at 10 Hz or less than 1 kohm at 10 Hz.

In an example arrangement, the position of the electrodes with respect to the external body part of the subject when the electrode harness 2/electrode support 4 is mounted on the subject may be employed in EIT image reconstruction. The position of the electrodes may be determined in a number of ways.

One way is that the control unit 10 may be further configured to determine the position of the electrodes 6 with respect to the external body part of the subject when the electrode support 4 is mounted on the subject by providing electrical signals and measuring electrical signal responses using different combinations of the plurality of electrodes and determining impedance responses based on the provided electrical signals and the measured electrical signal responses. In principle, it is possible to determine the boundary positions of electrodes used for bioimpedance recording, in addition to tomographic imaging of the internal tissues. This may be achieved by a number of approaches which generally require assumptions about the impedance values of internal tissues. One example is given in an article by Rashid, A., Kim, B. S., Khambampati, A. K., Kim, S., and Kim, K. Y. (2011) entitled “An oppositional biogeography-based optimization technique to reconstruct organ boundaries in human thorax using electrical impedance tomography”, Physiological Measurement, 32: 767-796 (the contents of which are incorporated herein in their entirety for all purposes).

In an additional or alternative way, the control unit 10 may be configured to determine the position of the electrodes 6 with respect to the external body part of the subject when the electrode support 4 is mounted on the subject by determining distances between electrodes of the plurality of electrodes 6. For example, where the electrode support 4 is made of flexible elasticated/stretchable material and the connectors 8 to the electrodes 6 comprise inbuilt or embedded tracks (e.g. conductive layers formed by vapour deposition, sputtering or similar method as discussed above or wires embedded in the electrode support 4 or other methods as discussed above), the distance between electrodes (e.g. the distance between neighbouring electrodes in a ring) once the electrode support 4 is mounted on the subject can be determined based on the length of each connector 8 to the respective electrode. The length can be calculated by determining the resistance of the connector 8.

In another additional or alternative way, the external computing device 6 may be configured to determine the position of the electrodes 6 with respect to the external body part of the subject when the electrode support 4 is mounted on the subject based on photogrammetry using one or more images obtained of the electrode harness 2 when mounted on the subject, or laser based time-of-flight measurements. When the external computing device 16 comprises a device with a camera or other imaging device (e.g. a camera of a smart phone or mobile phone or tablet or similar device), the one or more images may be taken by the camera or other imaging device of the external computing device 16 from different positions. The position of each electrode may be calculated using the technique of photogrammetry in which positions of identified objects may be calculated by triangulation of identified points from different camera positions. Additionally or alternatively, the position of electrodes may be determined by other optical methods such as a laser based time-of-flight approach. Determining the positions of the electrodes 6 once the electrode support 4 is mounted on the subject facilitates image reconstruction using EIT. For example, where a model of the external body part is used for EIT reconstruction, determining the position of the electrodes in 3D, enables the position of the electrodes to be placed on the computer model to improve image quality obtained by the reconstruction.

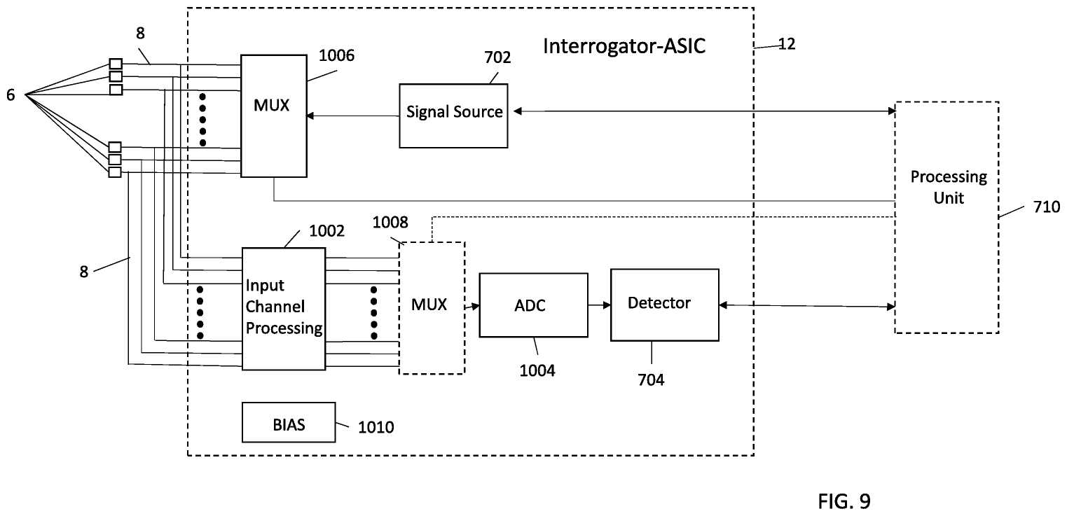

The interrogator 12 may be implemented in an ASIC or as one or more discrete integrated circuits mounted in a single package. FIG. 9 is a block schematic diagram showing some (not all) components of an example implementation of the interrogator 12 in an ASIC. Like components to those of FIG. 6 are referred to by the same reference numeral. The signal source 702 is coupled to the plurality of electrodes 6 via a multiplexer or switch 1006 (which performs the same multiplexing function as discussed above with reference to the multiplexer 706 in FIG. 6). The electrical signal response from each electrode of the plurality of electrodes 6 is coupled to a respective input channel of input channel processing bock 1002. The input channel processing block 1002 thus has a plurality of input channels corresponding to the plurality of electrodes 6 and may comprise components such as one or more amplifiers, low pass filter and buffer for each of the input channels. In an example arrangement shown in FIG. 9, after being processed by the input channel processing block 1002, each input channel may be coupled to a multiplexer 1008 before coupling to an analog-to-digital converter (ADC) 1004. The output of the ADC 1004 is provided to the detector 704 which outputs the measured response signals. As discussed above with reference to FIG. 6, a processing unit, such as the processing unit 710 of FIG. 6 (e.g. a MCU) is coupled at least to the multiplexer 1006, the input channel processing block 1002 and the multiplexer 1008 to control the operations of these blocks. In another example implementation, each input channel may have its own ADC and so the multiplexer 1008 is not required. In other implementations, there may be fewer record instrumentation amplifiers than electrodes (i.e. each input channel does not have an instrumentation amplifier) and so in such implementations, a multiplexer will be provided between the electrode or buffer amplifier and the fewer instrumentation amplifiers. The arrangement shown in FIG. 9 is therefore just one of many possible arrangements. The interrogator may also comprise bias block 1010 for supporting the analog blocks of the interrogator. Implementing the interrogator with multiple input channels on an ASIC facilitates the reduction in cost of the electrode harness 2 and also the size of the electronics required to carry out EIT on the harness 2. Having low cost and compact circuitry for carrying out EIT with multiple channels, ensures that a low cost electrode harness for carrying out EIT to provide sufficient quality images can be produced. Such low cost electrode harnesses are also easy to use which means they can be used widely outside of the research environment (e.g. by individual consumers).

In the foregoing description of the disclosure, reference has been made to particular examples. It will, however, be evident that various modifications and changes may be made to the description without departing from the scope of the invention as set forth in the appended claims.

1. An electrode harness for use in carrying out Electrical Impedance Tomography (EIT) on a human or animal subject, the electrode harness comprising:
an electrode support;
a plurality of electrodes on the electrode support for contacting a surface of an external body part of the human or animal subject;
a plurality of connectors, each connector coupled to a respective electrode of the plurality of electrodes; and
a control unit comprising:
an interrogator for coupling to the plurality of electrodes via the plurality of connectors, the interrogator being configured to selectively provide electrical signals to multiple groups of electrodes of the plurality of electrodes and for measuring, in response to the provided electrical signals, electrical response signals at at least some electrodes of the plurality of electrodes; and
communication circuitry for transmitting information associated with the electrical response signals to an external computing device.

2. The electrode harness of claim 1, wherein the control unit is integrated with the electrode support.

3. The electrode harness of claim 1, wherein the plurality of electrodes are on the electrode support such that, in use, the plurality of electrodes are arranged to form a plurality of rings around the electrode support and each ring comprises more than one electrode.

4. The electrode harness of claim 3, wherein the interrogator is configured to measure, in response to electrical signals being provided to electrodes of a particular ring, electrical response signals at electrodes of the particular ring.

5. The electrode harness of claim 1,
wherein the external body part is a chest of a subject and the electrode support is configured for mounting on the chest of the subject,
wherein the electrode support comprises a front portion and a back portion, and
wherein the plurality of electrodes are on the front portion and the back portion, the control unit is on one of the front portion or the back portion and each connector of the plurality of connectors extend from a respective electrode to the interrogator over the front portion and/or the back portion.

6. The electrode harness of claim 5, wherein the plurality of electrodes are arranged in at least two rings around the electrode support and each ring comprises at least eight electrodes.

7. The electrode harness of claim 1, wherein each electrode of the plurality of electrodes is configured to be a self-abrading electrode.

8. The electrode harness of claim 1, wherein each electrode comprises:
an electrode mount extending from a surface of the electrode support to provide an electrode mounting surface at an end furthest from the surface; and
a conductive pad formed on at least a portion of the electrode mounting surface, the conductive pad being electrically coupled to the respective connector.

9. The electrode harness of claim 8, wherein at least a portion of the electrode mounting surface is serrated, and
wherein the conductive pad is formed on at least a portion of the serrated electrode mounting surface.

10. The electrode harness of claim 8, wherein the electrode mount has a flat conical shape, and
wherein the electrode mounting surface is at least part of a flat surface of the flat conical shaped electrode mount.

11. The electrode harness claim 8, wherein at least a portion of the conductive pad is covered with a conductive adhesive material.

12. The electrode harness claim 8, wherein the electrode mount comprises the same material as the surface of the electrode support.

13. The electrode harness of claim 1, wherein the electrode support is formed from an elasticated or stretchable material.

14. The electrode harness of claim 1, further comprising an insulator layer formed over at least the connectors.

15. The electrode harness of claim 1, wherein the interrogator comprises:
a signal source for selectively providing electrical signals to the multiple groups of electrodes; and
a detector for measuring, in response to the provided electrical signals, electrical response signals at at least some electrodes of the plurality of electrodes.

16. The electrode harness of claim 1, wherein the control unit is further configured to determine a position of the electrodes with respect to the external body part of the subject when the electrode support is mounted on the subject by performing operations comprising:
providing electrical signals and measuring electrical signal responses using different combinations of the plurality of electrodes and determining impedance responses based on the provided electrical signals and the measured electrical signal responses; or
by determining distances between electrodes of the plurality of electrodes by measuring resistance of the connectors of the electrodes.

17. The electrode harness of claim 1, wherein the control unit is further configured to determine impedance responses of part of the subject within the external body based on the provided electrical signals and the measured electrical response signals and wherein the information transmitted to the external computing device includes the impedance responses for EIT processing at the external computing device.

18. The electrode harness claim 1, wherein the control unit further comprises an EIT processing module for carrying out an EIT reconstruction of impedance responses based on the provided electrical signals and the measured electrical response signals to derive electrical properties relating to part of the subject within the external body part, and
wherein the information provided to the external computing device includes the electrical properties derived from the EIT reconstruction.

19. The electrode harness of claim 1, wherein the control unit is configured to carry out an electrode contact quality test for each electrode of the plurality of electrodes when the electrode support is mounted on the subject to determine, for each tested electrode, whether a contact impedance between the tested electrode and the surface of the external body part of the subject meets a predetermined criteria and to provide, for each tested electrode, contact quality test result information for transmission by the communication circuitry to the external computing device.

20. The electrode harness of claim 19, wherein the control unit is configured to carry out the electrode contact quality test when the electrode support is mounted on the subject by, for each tested electrode by performing operations comprising:
measuring the contact impedance of the tested electrode;
comparing the measured contact impedance with a threshold impedance; and
when the measured contact impedance is greater than the threshold impedance, identifying the tested electrode as having a poor contact quality,
wherein when at least one tested electrode of the plurality of electrodes is identified as having a poor contact quality, the control unit is configured to provide contact quality test result information identifying the at least one tested electrode having poor contact quality for transmission by the communication circuitry to the external computing device.

21. A system comprising:
an electrode harness claim 1 for use in carrying out Electrical Impedance Tomography (EIT) on the human or animal subject; and
an external computing device for receiving information associated with the electrical response signals transmitted by the communication circuitry of the electrode harness,
wherein the external computing device comprises:
an EIT processing module for carrying out an EIT reconstruction of impedance responses based on the information associated with the electrical response signals received from the communication circuitry to derive electrical properties relating to part of the subject within the external body part; and
a user interface for providing information to a user relating to the derived electrical properties.

22. The system of claim 21, wherein the EIT processing module is configured to determine one or more parameters based on the derived electrical properties, and wherein the user interface is a display and the external computing device is configured to display at least one of the one or more parameters on the display of the external computing device.

23. The system of claim 21, wherein the EIT processing module is configured to reconstruct the impedance responses into a tomographic image of the electrical properties and wherein the user interface is a display and the external computing device is configured to display the tomographic image on the display of the external computing device.

24. The system claim 21, wherein the control unit is configured to carry out an electrode contact quality test for each electrode of the plurality of electrodes when the electrode support is mounted on the subject to determine, for each tested electrode, whether a contact impedance between the tested electrode and the surface of the external body part of the subject meets a predetermined criteria and to provide contact quality test result information for transmission by the communication circuitry to the external computing device, and
wherein the user interface of the external computing device is a display and the external computing device is configured to display a representation of contact quality of each of the plurality of electrodes based on the contact quality test result information.

25. The system of claim 21, wherein the external computing device is configured to determine positions of the electrodes with respect to the external body part of the subject when the electrode support is mounted on the subject based on information provided by the communication circuitry of the electrode harness.

26. The system of claim 21, wherein the external computing device is configured to determine positions of the electrodes with respect to the external body part of the subject when the electrode support is mounted on the subject based on photogrammetry using one or more images obtained of the electrode harness when mounted on the subject, or laser based time-of-flight measurements.

27. A method for determining electrical properties relating to part of a human or animal subject using Electrical Impedance Tomography (EIT), the method comprising:
mounting an electrode harness comprising a plurality of electrodes on an electrode support and a control unit on the subject such that at least some of the plurality of electrodes contact a surface of an external body part of the subject;
selectively providing electrical signals, from an interrogator of the control unit coupled to the plurality of electrodes via a plurality of connectors, to multiple groups of electrodes of the plurality of electrodes;
measuring, by the interrogator of the control unit, in response to the provided electrical signals, electrical response signals at at least some electrodes of the plurality of electrodes;
carrying out an EIT reconstruction of impedance responses based on the provided electrical signals and the measured electrical response signals to derive electrical properties relating to part of the subject within the external body part;
receiving, at the external computing device, the electrical properties relating to part of the subject within the external body part; and
providing, by a user interface of the external computing device, information to a user based on the derived electrical properties.

28. The method of claim 27, wherein carrying out an EIT reconstruction of impedance responses is performed by the control unit and the method further comprises:
transmitting, by communication circuitry of the control unit, information including the electrical properties to the external computing device.

29. The method of claim 27, wherein carrying out an EIT reconstruction of impedance responses is performed by the external computing device and the method further comprises:
transmitting, by communication circuitry of the control unit, information associated with the electrical response signals to the external computing device.

30. The method of claim 27, wherein the user interface of the external computing device is a display, the method further comprising:
determining one or more parameters based on the derived electrical properties, the one or more parameters relating to functioning of the part of the subject within the external body part; and
displaying at least one of the one or more parameters on the display of the external computing device.

31. The method claim 27, wherein the user interface of the external computing device is a display, the method further comprising:
determining, for each tested electrode and when the electrode support is mounted on the subject, whether a contact impedance between the tested electrode and the surface of the external body part of the subject meets a predetermined criteria;
providing, by the control unit to the external computing device, contact quality test result information based on determining whether a contact impedance between the tested electrode and the surface of the external body part of the subject meets a predetermined criteria; and
displaying on the display of the external computing device a representation of contact quality of each of the plurality of electrodes based on the contact quality test result information.

32. The method of claim 27, further comprising:
determining a position of the electrodes with respect to the external body part of the subject when the electrode support is mounted on the subject.
